(19) United States
(12) Patent Application Publication
(10) Pub. No.: US 2009/0240278 A1
(43) Pub. Date: Sep. 24, 2009

(54) DEVICE FOR PLACING AN OCCLUSION REMOVING STRUCTURE IN A TUBULAR STRUCTURE WITH A SUBSTANTIALLY CLOSED DISTAL END

(76) Inventor: Harvey L. Deutsch, Los Angeles, CA (US)

Correspondence Address: SHELDON MAK ROSE & ANDERSON PC, 100 Corson Street, Third Floor, PASADENA, CA, 91103-3842, US

(21) Appl. No.: 11/574,865

(22) PCT Filed: Feb. 15, 2007
(86) PCT No.: PCT/US2007/062263

§ 371 Date: Mar. 7, 2007

Related U.S. Application Data
(60) Provisional application No. 60/774,705, filed on Feb. 17, 2006.

(51) Int. Cl. A61M 29/00

(52) U.S. Cl. 606/198

(57) ABSTRACT

A device for placing the distal end of an occlusion removing structure at or near the distal end of a tubular structure with a substantially closed distal end. A method for placing the distal end of an occlusion removing structure into a tubular structure with a substantially closed distal end, where placing results in the distal end of the occlusion removing structure being positioned at or near the distal end of the tubular structure.

### CROSS-REFERENCE TO RELATED APPLICATION

The present Application claims the benefit of U.S. Provisional Patent Application 60/774,705 titled “Device for Placing an Occlusion Removing Structure in a Tubular Structure with a Substantially Closed Distal End,” filed Feb. 17, 2006, the contents of which are incorporated in this disclosure by reference in their entirety.

### BACKGROUND

There are a large variety of tubular structures comprising a central axial lumen and a substantially closed distal end used in medical applications and in non-medical commercial and industrial applications. Examples of such tubular structures used in medical applications include catheters and drains, such as, for example, Foley catheters. In many such applications, the tubular structure is prone to collecting occlusive debris within the central lumen of the tubular structure that occludes the tubular structure, causing a decrease or cessation of function of the tubular structure, and necessitating that the occlusion be removed or that the tubular structure be replaced.

In medical applications in particular, replacing a tubular structure can be very costly, and can put a patient at increased risk of infection. Therefore, it is often preferable to remove the occlusion from within the central lumen of the tubular structure while maintaining the tubular structure in position. In some applications, the occlusion can be removed by flushing the central lumen of the tubular structure with a liquid, thereby moving the occlusion out of the tubular structure through drainage holes in the side of the tubular structure. In many applications, however, flushing the occlusion out of the tubular structure is not appropriate, such as, for example, when using a tubular structure to drain a cavity or space within the human body.

Therefore, there is needed a device for placing an occlusion removing structure within a tubular structure with a substantially closed distal end, such that when the tubular structure becomes occluded, the occlusion removing structure can be withdrawn from the tubular structure proximally, thereby removing the occlusion proximally rather than distally while maintaining the tubular structure in position. Further, there is needed a method for unoccluding a tubular structure with a substantially closed distal end.

### SUMMARY

According to one embodiment of the present invention, there is provided a device for placing an occlusion removing structure into a tubular structure with a substantially closed distal end. The device comprises a) a proximal end, a distal end, and an intermediate portion between the proximal end and the distal end; b) an occlusion removing structure comprising a proximal end, and a distal end with a tip, and further comprising an elongated tubular mesh comprising a proximal end, a distal end, an axial length between the proximal end and the distal end, an outer surface, an inner surface and a central lumen cross-sectional area defined by the inner surface; where the tubular mesh exists in two states: i) a radially expanded and axially contracted first state, where the central lumen cross-sectional area is a first central lumen cross-sectional area, and the axial length is a first axial length; and ii) a radially contracted and axially expanded second state, where the central lumen cross-sectional area is a second central lumen cross-sectional area, and the axial length is a second axial length; and where the first central lumen cross-sectional area is greater than the second central lumen cross-sectional area, and the first axial length is less than the second axial length; c) a distracting structure for distracting the occlusion removing structure from the radially expanded and axially contracted first state to the radially contracted and axially expanded second state; and d) an attaching and locking structure for attaching and locking the occlusion removing structure to the distracting structure non-integrally, and for reversibly locking the occlusion removing structure in the radially contracted and axially expanded second state; and where the occlusion removing structure, distracting structure, and attaching and locking structure are all non-integral with respect to each other.

In one embodiment, the occlusion removing structure further comprises a proximal end piece at the proximal end of the occlusion removing structure, and a cap comprising the tip at the distal end of the occlusion removing structure. In another embodiment, the occlusion removing structure further comprises a proximal end piece at the proximal end of the occlusion removing structure; and the attaching and locking structure mates with the proximal end piece of the occlusion removing structure to allow the occlusion removing structure to be removed from a tubular structure while keeping the tubular structure in position.

In another embodiment, the distracting structure comprises: a) a proximal end comprising a first connector; and b) a distal end comprising an elongated tube or rod comprising a proximal end, a distal end, an axial length defined by the distance between the proximal end and the distal end, and an outer surface defining an outer circumference; and the elongated tube or rod further comprises a material suitable for forcing the tubular mesh into the radially contracted and axially expanded first state by increasing the axial length of the tubular mesh from the first axial length to the second axial length; and the proximal end of the elongated tube is joined to the first connector. In a preferred embodiment, the tube or rod further comprises two segments, a proximal segment and a distal segment; and the distal segment is relatively more laterally flexible than the proximal segment.

In another embodiment, the attaching and locking structure comprises two non-integral sections, a first section and a second section; and the first section comprises: i) a proximal end comprising a second connector for mating with the first connector of the distracting structure; and ii) a distal end. In a preferred embodiment, the distal end of the first section of the attaching and locking structure comprises a rigid tube comprising a proximal end, a distal end, an axial length between the proximal end and the distal end, an outer surface, an inner surface, and a central lumen defined by the inner surface. In another preferred embodiment, the second section of the attaching and locking structure comprises a proximal end and a distal end; and the proximal end of the second section comprises a third connector for mating with the distal end of the second connector of the first section. In a preferred embodiment, the occlusion removing structure further comprises a proximal end piece at the proximal end of the occlusion removing structure; the second section of the attaching and locking structure comprises a proximal end and a distal end; the distal end of the second section comprises two legs that are split axially from near the proximal end, through the distal end of the second section; and the legs form a fourth connector configured to mate with the proximal end piece of the occlusion removing structure.

According to another embodiment of the present invention, there is provided a method for placing an occlusion removing structure into a tubular structure with a proximal end, a substantially closed distal end and a central lumen. The method comprises: a) providing a device according to the present invention in the radially contracted and axially expanded second state; b) placing the occlusion removing structure of the device in the tubular structure; and c) allowing the occlusion removing structure to assume the radially expanded and axially contracted first state.

In one embodiment, placing the occlusion removing structure of the device comprises inserting the distal end of the device into the proximal end of the tubular structure, and advancing the distal end of the device through the central lumen of the tubular structure until the occlusion removing structure is entirely within the tubular structure, or until the distal end of the tubular structure contacts the substantially closed distal end of the tubular structure. In another embodiment, the distracting structure comprises, a proximal end comprising a first connector and a distal end; the attaching and locking structure comprises two non-integral sections, a first section and a second section; the first section comprises, i) a proximal end comprising a second connector for mating with the first connector of the distracting structure; and ii) a distal end; and the method further comprises disconnecting the first connector from the second connector, and withdrawing the distracting structure from the device while maintaining at least a portion of the tubular mesh within the tubular structure, thereby converting the tubular mesh from the radially contracted and axially expanded second state to the radially expanded and axially contracted first state. In another embodiment, the occlusion removing structure further comprises a proximal end piece at the proximal end of the occlusion removing structure; the distracting structure comprises, a proximal end comprising a first connector and a distal end; the attaching and locking structure comprises two non-integral sections, a first section and a second section; the first section comprises, i) a proximal end comprising a second connector for mating with the first connector of the distracting structure; and ii) a distal end; the second section of the attaching and locking structure comprises a proximal end comprising a third connector for mating with the distal end of the second connector of the first section and a distal end; the distal end of the second section comprises two legs that are split axially from near the proximal end, through the distal end of the second section; and the legs form a fourth connector configured to mate with the proximal end piece of the occlusion removing structure; where the method further comprises disconnecting the second connector from the third connector and withdrawing the first section of the attaching and locking structure from the second section of the attaching and locking structure, thereby allowing the legs of the second section to contract radially and unlock the fourth connector from the proximal end piece of the occlusion removing structure; and where the method further comprises removing the second section of the attaching and locking structure from the occlusion removing structure, thereby leaving the occlusion removing structure in the tubular structure. In another embodiment, the occlusion removing structure further comprises a proximal end piece at the proximal end of the occlusion removing structure; and the method further comprises separating the tubular mesh from the proximal end piece.

In another embodiment, the method further comprises allowing the tubular structure to become occluded with occluding material, and then withdrawing the occlusion removing structure from the tubular structure after the tubular structure becomes occluded with occluding material. In a preferred embodiment, the occlusion removing structure further comprises a proximal end piece at the proximal end of the occlusion removing structure; the attaching and locking structure comprises two non-integral sections, a first section and a second section; the first section comprises, i) a proximal end comprising a second connector; and ii) a distal end; the second section of the attaching and locking structure comprises a proximal end comprising a third connector for mating with the distal end of the second connector of the first section and a distal end; the distal end of the second section comprises two legs that are split axially from near the proximal end, through the distal end of the second section; and the legs form a fourth connector configured to mate with the proximal end piece of the occlusion removing structure; and; withdrawing the occlusion removing structure from the tubular structure after the tubular structure becomes occluded with occluding material comprises: d) inserting the fourth connector of the second section of the attaching and locking structure into the proximal end piece of the occlusion removing structure; e) then advancing the distal end of the first section of the attaching and locking structure though the proximal end of the second section, thereby radially expanding the legs of the second section and locking the fourth connector to the proximal end piece of the occlusion removing structure; f) connecting the second connector of the first section to the third connector of the second section; and g) retracting the occlusion removing structure, along with the attaching and locking structure, proximally, thereby, holding the occluding material within the tubular mesh as the occlusion removing structure is withdrawn from the tubular structure.

In one embodiment, the tubular structure is within a space or cavity within a human or within a non-human animal. In another embodiment, the space or cavity is created by a surgical procedure. In another embodiment, the space or cavity is selected from the group consisting of the abdominal cavity, the bladder, the intestines, the intracranial cavity, the mediastinum, the nasal passages, the stomach, the renal pelvis and the ureter. In another embodiment, the space or cavity is created during a surgical procedure on the back, breast, chest, head, hip or vertebral column. In another embodiment, the tubular structure is selected from the group consisting of an abscess drainage catheter, a biliary tube, a biliary stents, a chest tube, a decompression catheter, a feeding tube, a gastrointestinal decompression catheter, a gastrostomy tube, a jejunostomy tube, a mediastinal tube, a nasogastric tube, a nephrostomy catheter, a percutaneous drainage catheter, a peritoneal dialysis catheter, a vascular catheter, a venous hemodialysis catheter, a post-surgical drain and a ventriculostomy tube.

According to another embodiment of the present invention, there is provided a kit for placing an occlusion removing structure in a tubular structure with a substantially closed distal end. In one embodiment, the kit comprises a device according to the present invention, and instructions on how to use the device. In another embodiment, the kit comprises a device for placing an occlusion removing structure into a tubular structure with a substantially closed distal end, and instructions on how to use the device, where the instructions disclose the steps in a method according to the present invention

### FIGURES

These and other features, aspects and advantages of the present invention will become better understood with regard to the following description, appended claims, and accompanying figures where:

FIG. 1 is a top perspective view of one embodiment of a device according to the present invention for placing an occlusion removing structure into a tubular structure with a substantially closed distal end;

FIG. 2 is an exploded, top perspective view of the proximal end of the device shown in FIG. 1;

FIG. 3 is a partial, top perspective view of one embodiment of an occlusion removing structure (first component) of the device shown in FIG. 1 and FIG. 2;

FIG. 4 is a close-up, top perspective view of one embodiment of the proximal end of the occlusion removing structure;

FIG. 5 is a partial, top perspective view of one embodiment of a distracting structure (second component) of the device shown in FIG. 1 and FIG. 2; and

FIG. 6 is a partial, cross-sectional, top perspective view of one embodiment of an attaching and locking structure (third component) of the device shown in FIG. 1 and FIG. 2.

### DESCRIPTION

According to one embodiment of the present invention, there is provided a device for placing the distal end of an occlusion removing structure at or near the distal end of a tubular structure with a substantially closed distal end. According to another embodiment of the present invention, there is provided a method for placing the distal end of an occlusion removing structure into a tubular structure with a substantially closed distal end, where placing results in the distal end of the occlusion removing structure being positioned at or near the distal end of the tubular structure. In a preferred embodiment, the method comprises providing a device according to the present invention. The device and method prolong the useful life of a tubular structure, such as a medical drain, and obviate the need for replacing the tubular structure when it becomes occluded, thereby decreasing the cost associated with drain replacement, and the risk of patient morbidity associated with drain replacement. Though the device of the present invention is presented primarily in the context of use with a medical tubular structure in this disclosure, the device can also be used for other purposes, as will be understood by those with skill in the art with reference to this disclosure. Further, though the device and method are disclosed primarily in the context of use with a tubular structure with a substantially closed distal end, the device and method can also be used with a tubular structure with a substantially open distal end as will be understood by those with skill in the art with reference to this disclosure. The device and method will now be presented in detail.

As used in this disclosure, the term “comprise” and variations of the term, such as “comprising” and “comprises,” are not intended to exclude other additives, components, integers or steps.

As used in this disclosure, two elements of a device are “integral” if they are joined together in a manner that does not allow separation of the two elements from one another by the user of the device without cutting through or destroying the function of one or both of the two elements.

As used in this disclosure, two elements of a device are “non-integral” if they are joined together in a manner to allow separation of the two elements from one another by the user of the device without cutting through or destroying the function of one or both of the two elements.

As used in this disclosure, the term “occlusion” means a mass or clog of occluding material within the central lumen of a tubular structure, which either partially or completely decreases the function of a tubular structure, and the term “occlude” and variations of the term, such as “occluded,” and “occluding,” refer to a process where an occlusion within the central lumen of a tubular structure either partially or completely decreases the function of a tubular structure. As will be understood by those with skill in the art with reference to this disclosure, debris being aspirated from inside a blood vessel left after removal of an embolus or plaque from the blood vessel wall by a therapy catheter does not decrease the function of an aspiration catheter aspirating the debris and, therefore, the debris within the aspiration catheter is not an occlusion within the aspiration catheter.

As used in this disclosure, the term “at or near the distal end of the tubular structure” and corresponding phrases means that the tip of the distal end of the occlusion removing structure is positioned within the distal most 10% of the axial length of the tubular structure. For example, if the axial length of the tubular structure that resides within the space or cavity being tubular structured is 10 cm, then after placing the occlusion removing structure, the tip of the distal end of the occlusion removing structure is positioned between 9 cm and 10 cm from the proximal end of the tubular structure, that is, the tip of the distal end of the occlusion removing structure is positioned within 1 cm of the distal end of the tubular structure.

As used in this disclosure, the term “substantially closed distal end” means that the distal end of the tubular structure is either completely closed or comprises an opening of a cross-sectional area that is suitable only for passing a guide wire through, such as comprising a hole at the distal end comprising a cross-sectional area of between 1 mm and 3 mm, or comprises an opening of a cross-sectional area that is 20% or less of the average cross-sectional area of the central lumen of the tubular structure.

All dimensions specified in this disclosure are by way of example only and are not intended to be limiting. Further, the proportions shown in these Figures are not necessarily to scale. As will be understood by those with skill in the art with reference to this disclosure, the actual dimensions of any device or part of a device disclosed in this disclosure will be determined by its intended use.

As depicted in the figures, the proximal direction of the components is shown to the left of the figure, and the distal direction of the components is shown to the right of the figure. All dimensions specified in this disclosure are by way of example only and are not intended to be limiting. Further, the proportions shown in these Figures are not necessarily to scale. As will be understood by those with skill in the art with reference to this disclosure, the actual dimensions of any device or part of a device disclosed in this disclosure will be determined by its intended use.

The devices of the present invention and their component parts comprise any suitable material for the intended purpose of the device, as will be understood by those with skill in the art with reference to this disclosure. For example, when used with a medical drain, the device will usually comprise one or more than one biocompatible material capable of being sterilized.

The devices of the present invention and their component parts can be constructed according to standard techniques, as will be understood by those with skill in the art with reference to this disclosure.

As will be understood by those with skill in the art with reference to this disclosure, the device and method for the present invention can be used for a variety of both medical and non-medical uses. In one example, with respect to medical uses, the device and method of the present invention can be used to place an occlusion removing structure within a tubular structure selected from the group consisting of an abscess drainage catheter, a biliary tube, a biliary stent, a chest tube, a decompression catheter, a feeding tube, a gastrointestinal decompression catheter, a gastrostomy tube, a jejunostomy tube, a mediastinal tube, a nasogastric tube, a nephrostomy catheter, a percutaneous drainage catheter, a peritoneal dialysis catheter, a vascular catheter, a venous hemodialysis catheter, a post-surgical drain and a ventriculostomy tube. In another example, with respect to non-medical uses, the device and method of the present invention can be used to place an occlusion removing structure within a tubular structure selected from the group consisting of a household drain, a sewer pipe and a fuel line, as well as other tubular structures used throughout the automotive and petrochemical industries, as will be understood by those with skill in the art with reference to this disclosure.

According to one embodiment of the present invention, there is provided a device for placing an occlusion removing structure into a tubular structure with a substantially closed distal end, where the occlusion removing structure comprises a distal end with a tip, where the tubular structure comprises a distal end, and where placing the occlusion removing structure results in placing the tip of the distal end of the occlusion removing structure at or near the distal end of the tubular structure. The device comprises means for removing an occlusion, means for distracting the means for removing an occlusion from a radially expanded and axially contracted first state to a radially contracted and axially expanded second state, and means for attaching and locking the means for removing an occlusion in a radially contracted and axially expanded second state to the means for distracting. The structures disclosed in this disclosure that correspond to the means for removing an occlusion, the means for distracting the means for removing an occlusion, and the means for attaching and locking the means for removing an occlusion will be understood by those with skill in the art with reference to this disclosure. In general, as disclosed in this disclosure, the means for removing an occlusion corresponds to the occlusion removing structure (first component) of the device, the means for distracting the means for removing an occlusion corresponds to the distracting structure (second component) of the device, and the means for attaching and locking the means for removing an occlusion corresponds to the attaching and locking structure (third component) of the device.

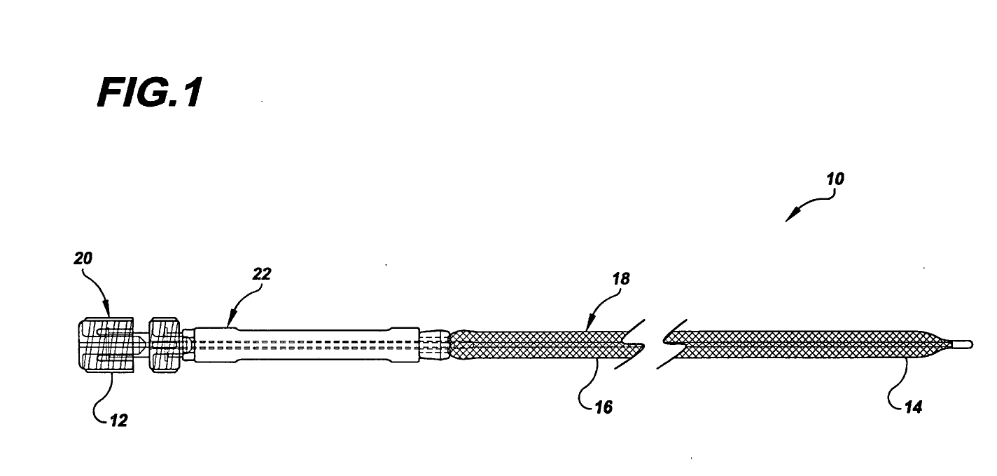

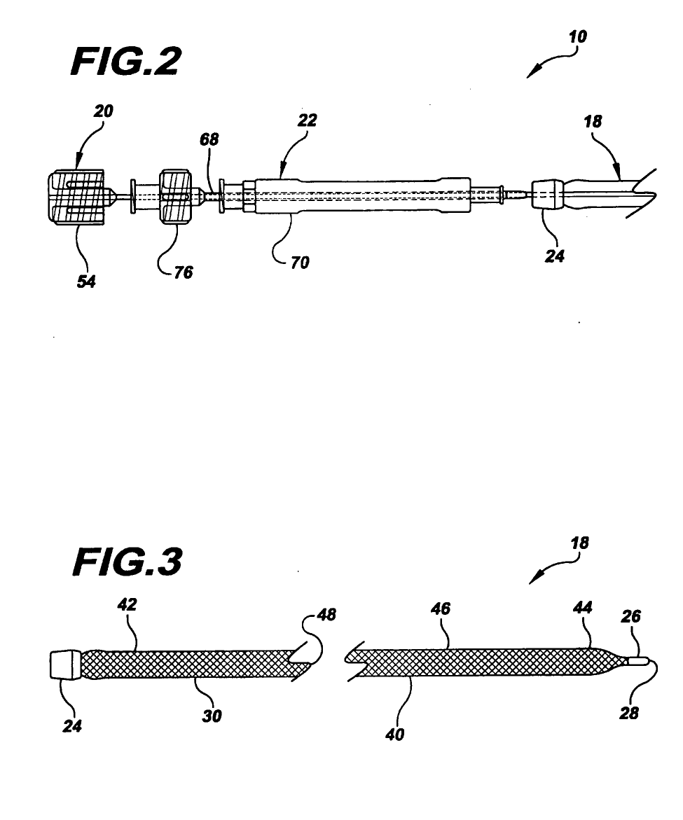

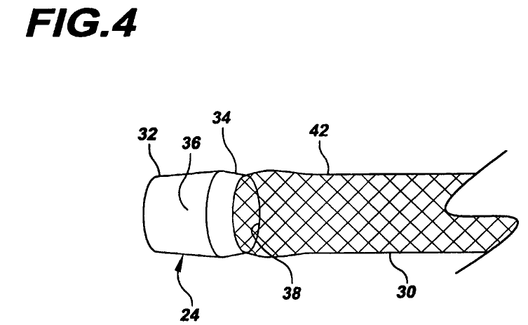

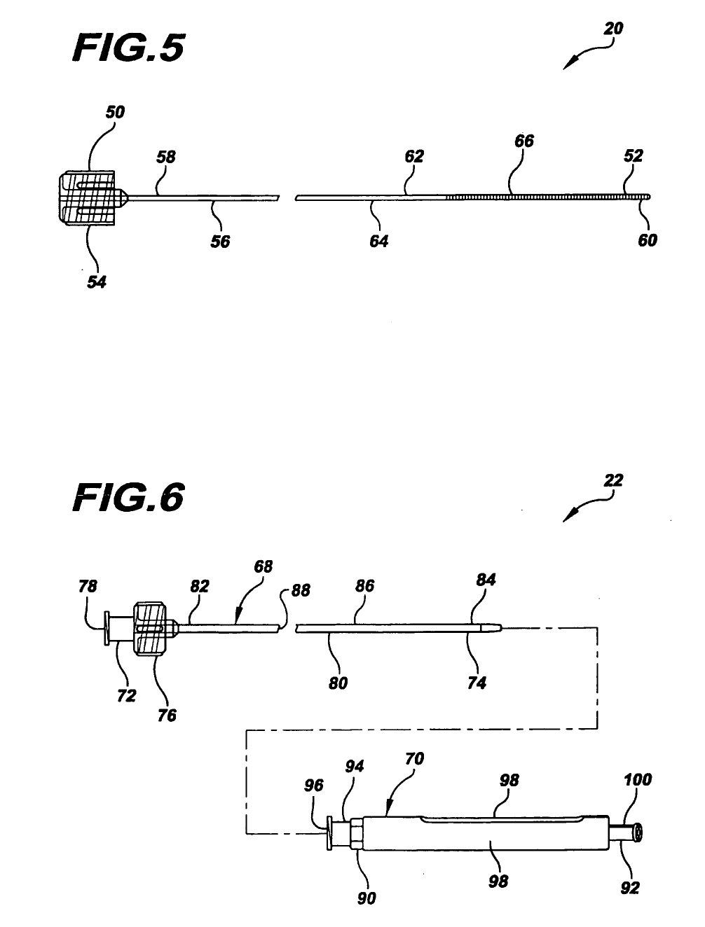

Referring now to FIG. 1, FIG. 2, FIG. 3, FIG. 4, FIG. 5 and FIG. 6, there are shown, respectively, a top perspective view of one embodiment of a device according to the present invention for placing an occlusion removing structure into a tubular structure with a substantially closed distal end (FIG. 1); an exploded, top perspective view of the proximal end of the device shown in FIG. 1 (FIG. 2); a partial, top perspective view of one embodiment of an occlusion removing structure (first component) of the device shown in FIG. 1 and FIG. 2 (FIG. 3); a close-up, top perspective view of one embodiment of the proximal end of the occlusion removing structure (FIG. 4); a partial, top perspective view of one embodiment of a distracting structure (second component) of the device shown in FIG. 1 and FIG. 2 (FIG. 5); a partial, cross-sectional, top perspective view of one embodiment of an attaching and locking structure (third component) of the device shown in FIG. 1 and FIG. 2 (FIG. 6). As can be seen, in one embodiment of the present invention, the device 10 comprises a proximal end 12, a distal end 14, and an intermediate portion 16 between the proximal end 12 and the distal end 14. The device 10 further comprises three components, an occlusion removing structure 18 (first component), a distracting structure 20 (second component) for distracting the occlusion removing structure 18 from a radially expanded and axially contracted first state to a radially contracted and axially expanded second state, and an attaching and locking structure 22 (third component) for attaching the occlusion removing structure 18 to the distracting structure 20 non-integrally, and for reversibly, locking the occlusion removing structure 18 in a radially contracted and axially expanded second state. The occlusion removing structure 18, distracting structure 20, and attaching and locking structure 22 are all non-integral with respect to each other.

Referring now to FIG. 1, FIG. 2, FIG. 3 and FIG. 4, the occlusion removing structure 18 comprises a proximal end comprising a proximal end piece 24, a distal end comprising a cap 26 with a tip 28 at the distal-most portion of the distal end, and an elongated tubular mesh 30 between the proximal end piece 24 and the cap 26. In one embodiment, as shown in FIG. 1, FIG. 2, FIG. 3 and particularly in FIG. 4, the proximal end piece 24 comprises a tube comprising a proximal end 32, a distal end 34, an axial length between the proximal end 32 and the distal end 34, an outer surface 36, an inner surface 38 and a cross-sectional area defined by the inner surface 38.

In a preferred embodiment, the tubular mesh 30 comprises a plurality of wires 40 forming a mesh, where each of the wires 40 crisscross a plurality of other wires 40, rather than comprising a plurality of wires that are merely spaced apart from one another or parallel to one another. In one embodiment, the tubular mesh 30 comprises between 5 and 50 wires 40. In another embodiment, the tubular mesh 30 comprises between 10 and 20 wires 40. In another embodiment, the tubular mesh 30 comprises between 12 and 15 wires 40.

In one embodiment, particularly as shown in FIG. 3 and FIG. 4, the tubular mesh 30 comprises a proximal end 42, a distal end 44, an axial length between the proximal end 42 and the distal end 44, an outer surface 46, an inner surface 48 and a central lumen cross-sectional area defined by the inner surface 48. The tubular mesh 30 exists in two states: 1) a radially expanded and axially contracted first state, where the central lumen cross-sectional area is a first central lumen cross-sectional area, and the axial length is a first axial length; and 2) a radially contracted and axially expanded second state, where the central lumen cross-sectional area is a second central lumen cross-sectional area, and the axial length is a second axial length. The first central lumen cross-sectional area is greater than the second central lumen cross-sectional area, and the first axial length is less than the second axial length.

As will be understood by those with skill in the art with reference to this disclosure, both the central lumen cross-sectional area and the axial length of the tubular mesh 30 will depend on the intended purpose of the device 10. By way of example only, in one embodiment, the first central lumen cross-sectional area is between 10 mm² and 100 mm². In another embodiment, the first central lumen cross-sectional area is between 15 mm² and 50 mm². In another embodiment, the first central lumen cross-sectional area is between 20 mm₂ and 30 mm². In another embodiment, the first axial length is between 5 cm and 50 cm. In another embodiment, the first axial length is between 10 cm and 30 cm. In one embodiment, the second central lumen cross-sectional area is between 1 mm₂ and 10 mm². In another embodiment, the second central lumen cross-sectional area is between 2 mm² and 5 mm². In another embodiment, the second axial length is between 10 cm and 100 cm. In another embodiment, the second axial length is between 20 cm and 60 cm.

The proximal end 42 of the tubular mesh 30 is integrally joined to the proximal end piece 24, such as, for example, by either mechanical means or by chemical means, such as, for example, by an adhesive such as an epoxy, or both by mechanical and by chemical means, by other suitable means such as spot welding or laser welding, as will be understood by those with skill in the art with reference to this disclosure.

In one embodiment, the wires 40 of the tubular mesh 30 comprise a material selected from the group consisting of cobalt-chromium-nickel-molybdenum-iron alloy specified by ASTM F1058 and ISO 5832-7, stainless steel and a ferrous alloy containing cobalt, chromium, nickel, molybdenum, manganese, carbon, and beryllium, such as Elgiloy® (Combined Metals of Chicago, L.L.C. Elk Grove Village, Ill. US).

In one embodiment, the cap 26 of the occlusion removing structure 18 comprises a small band that is crimped over the distal end of the wires 40 of the tubular mesh 30, bringing the distal end of the tubular mesh 30 to a tapered point. In a preferred embodiment, the cap comprises a material selected from the group consisting of cobalt-chromium-nickel-molybdenum-iron alloy specified by ASTM F1058 and ISO 5832-7, stainless steel and a ferrous alloy containing cobalt, chromium, nickel, molybdenum, manganese, carbon, and beryllium, such as Elgiloy® (Combined Metals of Chicago, L.L.C. Elk Grove Village, Ill. US).

Referring now to FIG. 1, FIG. 2 and FIG. 5, the device 10 further comprises a distracting structure 20 (second component) for distracting the occlusion removing structure 18 from a radially expanded and axially contracted first state to a radially contracted and axially expanded second state. The distracting structure 20 comprises a proximal end 50 and a distal end 52. The proximal end 50 of the distracting structure 20 comprises a first connector 54 configured to mate with the proximal end of the second connector, as disclosed below. In one embodiment, as shown, the first connector 54 comprises a luer lock-type “female” fitting with a threaded flange open distally. The first connector 54 can, however, be any suitable connector, as will be understood by those with skill in the art with reference to this disclosure.

The distal end 52 of the distracting structure 20 comprises an elongated tube or rod 56 comprising a proximal end 58, a distal end 60, an axial length defined by the distance between the proximal end 58 and the distal end 60, and an outer surface 62 defining an outer circumference. The proximal end 58 of the elongated tube 56 is joined to the first connector 54. In a preferred embodiment, the proximal end 58 of the elongated tube 56 is integrally joined to the first connector 54. In one embodiment, the tube or rod 56 comprises two segments, as shown in FIG. 4, a proximal segment 64 and a distal segment 66. When the elongated tube or rod 56 comprises two segments, the distal segment 66 is relatively more laterally flexible than the proximal segment 64.

The elongated tube or rod 56 comprises a material suitable for forcing the tubular mesh 30 into the radially contracted and axially expanded first state by increasing the axial length of the tubular mesh 30 from the first axial length to the second axial length, as will be understood by those with skill in the art with reference to this disclosure. In one embodiment, the elongated tube or rod 56 comprises a material selected from the group consisting of stainless steel and a flexible polymer. In one embodiment, the elongated tube or rod 56 comprises a material having a tensile strength of between 75 and 150 kpsi.

In one embodiment, the axial length of the elongated tube or rod 56 is at least 90% of the combined length of the second axial length of the tubular mesh 30 and the axial length of the attaching and locking structure 22, as disclosed below, when the attaching and locking structure 22 is attached to the proximal end piece 24 of the occlusion removing structure 18. In another embodiment, the axial length of the elongated tube or rod 56 is at least 95% of the combined length of the second axial length of the tubular mesh 30 and the axial length of the attaching and locking structure 22 when the attaching and locking structure 22 is attached to the proximal end piece 24 of the occlusion removing structure 18. In another embodiment, the axial length of the elongated tube or rod 56 is at least 99% of the combined length of the second axial length of the tubular mesh 30 and the axial length of the attaching and locking structure 22 when the attaching and locking structure 22 is attached to the proximal end piece 24 of the occlusion removing structure 18. In another embodiment, the axial length of the elongated tube or rod 56 is at least 100% of the combined length of the second axial length of the tubular mesh 30 and the axial length of the attaching and locking structure 22 when the attaching and locking structure 22 is attached to the proximal end piece 24 of the occlusion removing structure 18. In another embodiment, the axial length of the elongated tube or rod 56 is at least 105% of the combined length of the second axial length of the tubular mesh 30 and the axial length of the attaching and locking structure 22 when the attaching and locking structure 22 is attached to the proximal end of the occlusion removing structure 18. In another embodiment, the axial length of the elongated tube or rod 56 is at least 110% of the combined length of the second axial length of the tubular mesh 30 and the axial length of the attaching and locking structure 22 when the attaching and locking structure 22 is attached to the proximal end piece 24 of the occlusion removing structure 18.

Referring now to FIG. 1, FIG. 2 and FIG. 6, the device 10 further comprises an attaching and locking structure 22 (third component). The attaching and locking structure 22 serves two functions. First, the attaching and locking structure 22 mates with the proximal end piece 24 of the occlusion removing structure 18 to allow the occlusion removing structure 18 to be removed from a tubular structure while keeping the tubular structure in position. Second, the attaching and locking structure 22 serves to lock the occlusion removing structure 18 to the distracting structure 20, thereby maintaining the occlusion removing structure 18 in a radially contracted and axially expanded second state when the distracting structure 20 distracts the occlusion removing structure 18 from a radially expanded and axially contracted first state to the radially contracted and axially expanded second state.

The attaching and locking structure 22 comprises two non-integral sections, a first section 68 and a second section 70. As can be seen, the first section 68 comprises a proximal end 72 and a distal end 74. The proximal end 72 of the first section 68 comprises a second connector 76 for mating with the first connector 54 of the distracting structure 20, and for mating with the third connector, disclosed below, of the second section 70. The second connector 76 comprises a central axial lumen 78. In one embodiment, as shown particularly in FIG. 2 and FIG. 6, the proximal end of the second connector 76 comprises a “male” extension that is configured to mate with the threaded flange of the “female” fitting of the first connector 54. Further as shown particularly in FIG. 2 and FIG. 6, the distal end of the second connector 76 comprises a “female” fitting with a threaded flange open distally for mating with the third connector, disclosed below, of the second section 70. The second connector 76 can, however, be any suitable connector, as will be understood by those with skill in the art with reference to this disclosure.

The distal end 74 of the first section 68 of the attaching and locking structure 22 comprises a rigid tube 80 comprising a proximal end 82, a distal end 84, an axial length between the proximal end 82 and the distal end 84, an outer surface 86, an inner surface 88, and a central lumen defined by the inner surface 88. In a preferred embodiment, as shown, the distal end 84 of the rigid tube 80 is tapered.

The second section 70 of the attaching and locking structure 22 comprises a proximal end 90 and a distal end 92. The proximal end 90 of the second section 70 comprises a third connector 94 for mating with the distal end of the second connector 76 of the first section 68. The third connector 94 comprises a central axial lumen 96. In one embodiment, as shown, the third connector 94 comprises a “male” extension that is configured to mate with the threaded flange of the “female” fitting of the second connector 76. The third connector 94 can, however, be any suitable connector, as will be understood by those with skill in the art with reference to this disclosure.

The proximal end 90 of the second section 70 comprises two legs 98 that are split axially from near the proximal end 90, through the distal end 92 of the second section 70. The distal end of the legs 98 form a fourth connector 100 configured to mate with the proximal end piece 24 of the occlusion removing structure 18.

The three components of the device 10, the occlusion removing structure 18 (first component), the distracting structure 20 (second component), and the attaching and locking structure 22 (third component) are joined together non-integrally to form the assembled device 10 as follows. The fourth connector 100 of the second section 70 is mated to the proximal end piece 24 of the occlusion removing structure 18. The distal end 74 of the first section 68 is advanced completely through the central lumen of the third connector 94 of the second section 70 until the second connector 76 of the first section 68 mates with the third connector 94 of the second section 70, radially expanding the legs 98 and fourth connector 100 and, thereby, attaching the attaching and locking structure 22 to the occlusion removing structure 18. Next, the distal end 52 of the distracting structure 20 is advanced completely through the first section 68 from the proximal end 72 of the first section 68 through the distal end 74 of the first section 44, through the proximal end piece 24 of the occlusion removing structure 18 and through the central lumen of the tubular mesh 30, until the distal end 52 of the distracting structure 20 contacts the cap 26 of the occlusion removing structure 18, thereby distracting the occlusion removing structure 18 from the radially expanded and axially contracted first state to the radially contracted and axially expanded second state. Then, the first connector 54 of the distracting structure 20 is mated to the second connector 76, thereby locking the occlusion removing structure 18 in the second state, and creating an assembled device 10.

In another embodiment, the present invention is a kit for placing an occlusion removing structure in a tubular structure with a substantially closed distal end. The kit comprises a device according to the present invention, and further comprises instructions on how to use the device. In one embodiment, the instructions disclose the steps in a method according to the present invention.

In another embodiment, the present invention comprises a method for placing an occlusion removing structure into a tubular structure with a substantially closed distal end. The tubular structure has a proximal end, a distal end, and a wall comprising an outer surface and an inner surface defining an axial lumen. In one embodiment, the tubular structure further comprises one or more than one connector at the proximal end for attaching to a suction device, an irrigation device or both a suction device and an irrigation device. In a preferred embodiment, the tubular structure comprises one or more than one aperture in the wall.

In one embodiment, the method for placing an occlusion removing structure into a tubular structure with a substantially closed distal end comprises providing a device according to the present invention, and placing the occlusion removing structure of the device in the tubular structure. In another embodiment, the method comprises providing a device according to the present invention in the radially contracted and axially expanded second state, placing the occlusion removing structure of the device in the tubular structure; and allowing the occlusion removing structure to assume the radially expanded and axially contracted first state.

In a preferred embodiment, placing results in the distal end of the occlusion removing structure being positioned at or near the distal end of the tubular structure. In a preferred embodiment, the method comprises providing an assembled device for placing an occlusion removing structure in a tubular structure with a substantially closed distal end according to the present invention. Placing an occlusion removing structure in the tubular structure can be performed before the tubular structure becomes occluded, or can be performed after an occlusion has been removed from the tubular structure, such as, for example, to replace an occlusion removing structure that has been removed so that another occlusion can be removed from the tubular structure if the tubular structure becomes occluded again. The method will now be disclosed in detail.

First, the method comprises providing an assembled device for placing an occlusion removing structure in a tubular structure with a substantially closed distal end according to the present invention having the characteristics as disclosed in this disclosure. The device comprises an occlusion removing structure in a radially contracted and axially expanded second state, a distracting structure and an attaching and locking structure. In a preferred embodiment, the device provided is a device according to the present invention with all of the attributes disclosed in this disclosure. Then, the distal end of the device is inserted into the proximal end of the tubular structure, and the distal end of the device is advanced through the central lumen of the tubular structure until the occlusion removing structure is entirely within the tubular structure, or until the distal end of the tubular structure contacts the substantially closed distal end of the tubular structure.

Next, the method comprises disconnecting the first connector from the second connector, and withdrawing the distracting structure from the device, while maintaining at least a portion of the tubular mesh within the tubular structure, thereby converting the tubular mesh from the radially contracted and axially expanded second state to the radially expanded and axially contracted first state. In a preferred embodiment, the outer surface of the tubular mesh contacts at least part of the inner surface of the wall of the tubular structure when the tubular mesh is in the first state, thereby limiting the further radial expansion of the tubular mesh.

In one embodiment, the method further comprises disconnecting the second connector from the third connector and withdrawing the first section of the attaching and locking structure from the second section of the attaching and locking structure, thereby allowing the legs of the second section to contract radially and unlock the fourth connector from the proximal end piece of the occlusion removing structure. Then, the second section of the attaching and locking structure is removed from the occlusion removing structure, thereby leaving the occlusion removing structure in the tubular structure.

In one embodiment, after placing the occlusion removing structure, the proximal end of the tubular mesh extends proximally outside of the proximal end of tubular structure, and the method further comprises separating the tubular mesh from the proximal end piece, such as for example, by cutting the tubular mesh at or near the proximal end of the tubular mesh. In a preferred embodiment, the method further comprises attaching the cut end of the tubular mesh to a structure to secure the cut ends of the wires of tubular mesh.

In another embodiment, the method comprises allowing the tubular structure to become occluded with occluding material, and then withdrawing the occlusion removing structure from the tubular structure after the tubular structure becomes occluded with occluding material. In one embodiment, withdrawing the occlusion removing structure from the tubular structure after the tubular structure becomes occluded with occluding material comprises, first, inserting the fourth connector of the second section of the attaching and locking structure into the proximal end piece of the occlusion removing structure. Next, the distal end of the first section of the attaching and locking structure is advanced completely though the proximal end of the second section, thereby radially expanding the legs of the second section and locking the fourth connector to the proximal end piece of the occlusion removing structure. The second connector of the first section is then connected to the third connector of the second section. The occlusion removing structure, along with the attaching and locking structure, is then retracted proximally, distracting the tubular mesh and, thereby, holding the occluding material within the tubular mesh as the occlusion removing structure is withdrawn from the tubular structure.

In one embodiment, the tubular structure is within a space or cavity within a human or within a non-human animal. In another embodiment, the space or cavity is created by a surgical procedure. In another embodiment, the space or cavity is selected from the group consisting of the abdominal cavity, the bladder, the intestines, the intracranial cavity, the mediastinum, the nasal passages, the stomach, the renal pelvis and the ureter. In another embodiment, the space or cavity is created during a surgical procedure on the back, breast, chest, head, hip or vertebral column. In one embodiment, the tubular structure has become occluded with an occluding material within a space or cavity within a human or within a non-human animal.

Although the present invention has been discussed in considerable detail with reference to certain preferred embodiments, other embodiments are possible. Therefore, the scope of the appended claims should not be limited to the description of preferred embodiments contained in this disclosure.

What is claimed is:

1. A device for placing an occlusion removing structure into a tubular structure with a substantially closed distal end, the device comprising:
a) a proximal end, a distal end, and an intermediate portion between the proximal end and the distal end;
b) an occlusion removing structure comprising a proximal end, and a distal end with a tip, and further comprising an elongated tubular mesh comprising a proximal end, a distal end, an axial length between the proximal end and the distal end, an outer surface, an inner surface and a central lumen cross-sectional area defined by the inner surface;
where the tubular mesh exists in two states: i) a radially expanded and axially contracted first state, where the central lumen cross-sectional area is a first central lumen cross-sectional area, and the axial length is a first axial length; and ii) a radially contracted and axially expanded second state, where the central lumen cross-sectional area is a second central lumen cross-sectional area, and the axial length is a second axial length; and
where the first central lumen cross-sectional area is greater than the second central lumen cross-sectional area, and the first axial length is less than the second axial length;
c) a distracting structure for distracting the occlusion removing structure from the radially expanded and axially contracted first state to the radially contracted and axially expanded second state; and
d) an attaching and locking structure for attaching and locking the occlusion removing structure to the distracting structure non-integrally, and for reversibly locking the occlusion removing structure in the radially contracted and axially expanded second state; and
where the occlusion removing structure, distracting structure, and attaching and locking structure are all non-integral with respect to each other.

2. The device of claim 1, where the occlusion removing structure further comprises a proximal end piece at the proximal end of the occlusion removing structure, and a cap comprising the tip at the distal end of the occlusion removing structure.

3. The device of claim 2, where the occlusion removing structure further comprises a proximal end piece at the proximal end of the occlusion removing structure; and
where the attaching and locking structure mates with the proximal end piece of the occlusion removing structure to allow the occlusion removing structure to be removed from a tubular structure while keeping the tubular structure in position.

4. The device of claim 1, where the distracting structure comprises:
a) a proximal end comprising a first connector; and
b) a distal end comprising an elongated tube or rod comprising a proximal end, a distal end, an axial length defined by the distance between the proximal end and the distal end, and an outer surface defining an outer circumference; and
where the elongated tube or rod further comprises a material suitable for forcing the tubular mesh into the radially contracted and axially expanded first state by increasing the axial length of the tubular mesh from the first axial length to the second axial length; and
where the proximal end of the elongated tube is joined to the first connector.

5. The device of claim 4, where the tube or rod further comprises two segments, a proximal segment and a distal segment; and
where the distal segment is relatively more laterally flexible than the proximal segment.

6. The device of claim 1, where the attaching and locking structure comprises two non-integral sections, a first section and a second section;
where the first section comprises, i) a proximal end comprising a second connector for mating with the first connector of the distracting structure; and ii) a distal end.

7. The device of claim 6, where the distal end of the first section of the attaching and locking structure comprises a rigid tube comprising a proximal end, a distal end, an axial length between the proximal end and the distal end, an outer surface, an inner surface, and a central lumen defined by the inner surface.

8. The device of claim 6, where the second section of the attaching and locking structure comprises a proximal end and a distal end; and
where the proximal end of the second section comprises a third connector for mating with the distal end of the second connector of the first section.

9. The device of claim 6, where the occlusion removing structure further comprises a proximal end piece at the proximal end of the occlusion removing structure;
where the second section of the attaching and locking structure comprises a proximal end and a distal end;
where the distal end of the second section comprises two legs that are split axially from near the proximal end, through the distal end of the second section; and
where the legs form a fourth connector configured to mate with the proximal end piece of the occlusion removing structure.

10. A method for placing an occlusion removing structure into a tubular structure with a proximal end, a substantially closed distal end and a central lumen, the method comprising:
a) providing a device according to claim 1 in the radially contracted and axially expanded second state;
b) placing the occlusion removing structure of the device in the tubular structure; and
c) allowing the occlusion removing structure to assume the radially expanded and axially contracted first state.

11. The method of claim 10, where placing the occlusion removing structure of the device comprises inserting the distal end of the device into the proximal end of the tubular structure, and advancing the distal end of the device through the central lumen of the tubular structure until the occlusion removing structure is entirely within the tubular structure, or until the distal end of the tubular structure contacts the substantially closed distal end of the tubular structure.

12. The method of claim 10, where the distracting structure comprises, a proximal end comprising a first connector and a distal end;
where the attaching and locking structure comprises two non-integral sections, a first section and a second section;
where the first section comprises, i) a proximal end comprising a second connector for mating with the first connector of the distracting structure; and ii) a distal end; and
where the method further comprises disconnecting the first connector from the second connector, and withdrawing the distracting structure from the device while maintaining at least a portion of the tubular mesh within the tubular structure, thereby converting the tubular mesh from the radially contracted and axially expanded second state to the radially expanded and axially contracted first state.

13. The method of claim 10, where the occlusion removing structure further comprises a proximal end piece at the proximal end of the occlusion removing structure;
where the distracting structure comprises, a proximal end comprising a first connector and a distal end;
where the attaching and locking structure comprises two non-integral sections, a first section and a second section;
where the first section comprises, i) a proximal end comprising a second connector for mating with the first connector of the distracting structure; and ii) a distal end;
where the second section of the attaching and locking structure comprises a proximal end comprising a third connector for mating with the distal end of the second connector of the first section and a distal end;
where the distal end of the second section comprises two legs that are split axially from near the proximal end, through the distal end of the second section; and
where the legs form a fourth connector configured to mate with the proximal end piece of the occlusion removing structure;
where the method further comprises disconnecting the second connector from the third connector and withdrawing the first section of the attaching and locking structure from the second section of the attaching and locking structure, thereby allowing the legs of the second section to contract radially and unlock the fourth connector from the proximal end piece of the occlusion removing structure; and
where the method further comprises removing the second section of the attaching and locking structure from the occlusion removing structure, thereby leaving the occlusion removing structure in the tubular structure.

14. The method of claim 10, where the occlusion removing structure further comprises a proximal end piece at the proximal end of the occlusion removing structure; and
where the method further comprises separating the tubular mesh from the proximal end piece.

15. The method of claim 10, further comprising allowing the tubular structure to become occluded with occluding material, and then withdrawing the occlusion removing structure from the tubular structure after the tubular structure becomes occluded with occluding material.

16. The method of claim 15, where the occlusion removing structure further comprises a proximal end piece at the proximal end of the occlusion removing structure;
where the attaching and locking structure comprises two non-integral sections, a first section and a second section;
where the first section comprises, i) a proximal end comprising a second connector; and ii) a distal end;
where the second section of the attaching and locking structure comprises a proximal end comprising a third connector for mating with the distal end of the second connector of the first section and a distal end;
where the distal end of the second section comprises two legs that are split axially from near the proximal end, through the distal end of the second section; and
where the legs form a fourth connector configured to mate with the proximal end piece of the occlusion removing structure; and
where withdrawing the occlusion removing structure from the tubular structure after the tubular structure becomes occluded with occluding material comprises:
d) inserting the fourth connector of the second section of the attaching and locking structure into the proximal end piece of the occlusion removing structure;
e) then advancing the distal end of the first section of the attaching and locking structure though the proximal end of the second section, thereby radially expanding the legs of the second section and locking the fourth connector to the proximal end piece of the occlusion removing structure;
f) connecting the second connector of the first section to the third connector of the second section; and
g) retracting the occlusion removing structure, along with the attaching and locking structure, proximally, thereby, holding the occluding material within the tubular mesh as the occlusion removing structure is withdrawn from the tubular structure.

17. The method of claim 10, where the tubular structure is within a space or cavity within a human or within a non-human animal.

18. The method of claim 17, where the space or cavity is created by a surgical procedure.

19. The method of claim 17, where the space or cavity is selected from the group consisting of the abdominal cavity, the bladder, the intestines, the intracranial cavity, the mediastinum, the nasal passages, the stomach, the renal pelvis and the ureter.

20. The method of claim 17, where the space or cavity is created during a surgical procedure on the back, breast, chest, head, hip or vertebral column.

21. The method of claim 10, where the tubular structure is selected from the group consisting of an abscess drainage catheter, a biliary tube, a biliary stent, a decompression catheter, a feeding tube, a gastrointestinal decompression catheter, a gastrostomy tube, a jejunostomy tube, a mediastinal tube, a nasogastric tube, a nephrostomy catheter, a percutaneous drainage catheter, a peritoneal dialysis catheter, a vascular catheter, a venous hemodialysis catheter, a post-surgical drain and a ventriculostomy tube

22. A kit for placing an occlusion removing structure in a tubular structure with a substantially closed distal end, the kit comprising a device according to claim 1 and instructions on how to use the device.

23. A kit for placing an occlusion removing structure in a tubular structure with a substantially closed distal end, the kit comprising a device for placing an occlusion removing structure into a tubular structure with a substantially closed distal end, and instructions on how to use the device, where the instructions disclose the steps in a method according to claim 10.
